(19) United States
(12) Patent Application Publication
(10) Pub. No.: US 2021/0039955 A1
(43) Pub. Date: Feb. 11, 2021

(54) HEXAGONAL SILICA PLATELETS AND METHODS OF SYNTHESIS THEREOF

(71) Applicant: AGENCY FOR SCIENCE, TECHNOLOGY AND RESEARCH, Singapore (SG)

(72) Inventors: Gen YONG, Singapore (SG); Weiyin XU, Singapore (SG); Connie Kin Man LIU, Singapore (SG)

(21) Appl. No.: 16/071,074

(22) PCT Filed: Jan. 18, 2017
(86) PCT No.: PCT/SG2017/050025

§ 371 Date: Jul. 18, 2018

(30) Foreign Application Priority Data
Jan. 18, 2016 (SG) 10201600387Y

(51) Int. Cl. C01B 33/18; A61K 8/25; A61Q 17/04

(52) U.S. Cl. CPC C01B 33/18; B82Y 40/00; A61Q 17/04; A61K 8/25

(57) ABSTRACT

The present invention relates to a method for forming silica nanoparticles in an aqueous medium, the method comprising steps of providing a surfactant solution comprising a cationic surfactant in an aqueous medium; and mixing a silane source with the surfactant solution under pH conditions of about pH 5 to 8 for forming said silica nanoparticles. The present invention also relates to silica suspension comprising a plurality of hexagonally shaped silica platelets, said platelets being substantially monodisperse and having a width dimension from around 50 to 1000 nm, said platelets being suspended in an aqueous medium, wherein said aqueous medium comprises a single type of cationic surfactant. Furthermore, the invention relates to a silica nanoparticle prepared by reacting a silane source with a volumetric excess of a surfactant under pH of about 5 to about 8 in the presence of an aqueous solvent. Also, the present invention relates to a silica platelet obtainable by a method as disclosed herein.

### PRIORITY CLAIM

The present application claims priority to Singapore patent application number 10201600387Y.

### TECHNICAL FIELD

The present invention generally relates to silica structures and methods of preparing the same.

### BACKGROUND ART

Traditionally, silica nanoparticles are prepared by multi-step, sol-gel synthesis methods involving the hydrolysis of a silane source and the polycondensation of the hydrolysed silane source. Typically, an organic solvent (e.g. alcohol) is required for the dissolution of the silane source. Furthermore, the hydrolysis of a silane can be catalysed by a basic solution but more commonly, an acidic solution is used. In addition, the hydrolysed silane frequently requires a basic solution to catalyse the condensation reaction and/or the application of high temperature to sustain the condensation reaction. As such, multiple steps are involved and many reactants are required to obtain silica nanoparticles with the desired characteristics.

Moreover, in order to achieve high surface area in the resultant silica particles, surfactant-templating technique is employed which further complicates the preparation process. Most reported surfactant-templating processes require long reaction times of (up to 7 days or more) for the desired inorganic structure to be obtained. Furthermore, such processes often result in the formation of inorganic particles which are larger than 1 μm.

Furthermore, presently known processes for preparing template silica particles are unable to yield silica particles which are substantially monodispersed. In addition, current templating techniques tend to result in mesoporous silica particles instead of microporous silica particles. These may limit the potential applications of the silica structures.

There is therefore a need to provide alternative methods for preparing silica nanoparticles that addresses the above problems or achieves the desired properties, geometry or morphology. In particular, there is a need to provide an alternative method for producing silica nanoparticles, which is non-complex and which can be carried out under mild conditions.

### SUMMARY OF INVENTION

According to a first aspect, there is provided a method of forming silica nanoparticles in an aqueous medium, the method comprising steps of (a) providing a surfactant solution comprising a cationic surfactant in an aqueous medium; and (b) mixing a silane source with the surfactant solution from step (a) under pH conditions of about pH 5 to 8 for forming said silica nanoparticles.

The disclosed method may be characterized by the use of a single cationic surfactant for formation of the silica nanoparticles. Advantageously, the use of non-ionic surfactants may be avoided. Accordingly, in embodiments, the surfactant solution may consist essentially of a cationic surfactant as described herein.

Advantageously, the method may be performed in a substantially aqueous environment, rendering the method environmentally friendly. This may simplify the process of isolating or extracting the silica nanoparticles once formed. Steps associated with organic solvent separation and/or disposal are advantageously avoided. Also, the disclosed method is straightforward in that it requires relatively few reactants or additives. The disclosed method may be a single step, one-pot aqueous reaction. The disclosed method may be performed without pH variation or at substantially the same pH condition throughout. For instance, the addition of an acid or a basic medium is optional, unlike conventional sol-gel synthesis method which require acid hydrolysis. As a result, the disclosed method may result in less waste being generated.

The mild pH conditions of about pH 5 to 8 during the mixing step allows the preparation of the silica nanoparticles to be carried out in a safe and simple manner. Advantageously, the disclosed method avoids or eliminates the requirement for expensive processing equipment designed to be resistant to acidic and/or basic environments, which makes it easier to scale up for industrial processes.

The disclosed method may surprisingly be capable of forming three-dimensional silica nanoparticles such as hexagonal platelets, spheres or torus-like particles.

Advantageously, these three-dimensional silica nanoparticles may be substantially uniform in size or monodispersed. Advantageously, the hexagonal silica platelets may be useful for deploying the disclosed silica particles as deposition aids, substrates for array formation, as a clotting material, etc. Furthermore, the hexagonal silica platelets may be highly scattering in the UV/VIS spectral region and may be advantageously used as cosmetic whitening agents or as pigments in UV blocking/absorption compositions,

According to another aspect, there is provided a silica suspension comprising a plurality of hexagonally shaped silica platelets, said platelets being substantially monodisperse and having a width dimension from around 50 to 1000 nm, said platelets being suspended in an aqueous medium, wherein said aqueous medium comprises a single type cationic surfactant. The silica platelets may be prepared in accordance with the methods disclosed herein.

In a further aspect, there is provided a silica nanoparticles prepared by reacting a silane source with a volumetric excess of a surfactant under pH of about 5 to about 8 in the presence of an aqueous solvent.

In yet another aspect, there is provided a silica nanoparticles obtainable by a method as disclosed herein.

In another aspect, there is provided a cosmetic composition comprising a silica suspension or silica nanoparticles as disclosed herein.

In a further aspect, there is provided a method of providing UV shielding properties to a composition, comprising adding a silica suspension or silica nanoparticles as disclosed herein to said composition.

### Definitions

The following words and terms used herein shall have the meaning indicated:

As used herein, the expression “silica nanoparticles” is to be interpreted broadly to include three-dimensional silica particles which may be but not limited to hexagonal platelets, spheres, torus-like particles. These silica particles may be less than 2000 nm in at least one of the dimensions selected from the thickness, length, width or diameter. In the case of torus-like particles, the diameter may refer to the longest distance traversing the outermost circumference of the particle. Throughout the present disclosure, the expression “silica particles” or “silica nanoparticles” may be used interchangeably.

As used herein, the term “platelets” when used to describe silica platelets of the present invention refers to substantially hexagonal, three-dimensional structures. These three-dimensional hexagonal silica platelets may have a thickness dimension of around 1 nm to 100 nm. These hexagonal platelets may have a transverse length dimension of around 50 nm to 1000 nm. Throughout the present disclosure, the expression “silica plates” may be used interchangeably with the expression “silica platelets”

The term “aqueous medium” is to be interpreted broadly to include a liquid predominantly containing at least 50 wt. %, at least 60 wt. %, at least 70 wt. %, at least 80 wt. % or at least 90 wt. % water. The term aqueous medium may refer to substantially pure water.

As used herein, the term “aspect ratio” when used to describe silica nanoparticles, refers to a theoretical ratio of the measured surface area of the silica nanoparticles in a silica suspension to the measured volume of the silica nanoparticles in a silica suspension. The surface area of the silica nanoparticles may be measured e.g. by BET analysis. The theoretical volume may be calculated by measuring the dimensions (e.g. width, length, height) of the plate using, e.g., Atomic Force Microscopy (AFM). This mode of calculating the aspect ratio can be advantageously applied to any hexagonal plate having a width, height or length of any dimension or any torus-like particles having a diameter any dimension.

The term “cationic surfactant” is to be interpreted broadly to include a surfactant having a hydrophilic head group containing a positive net charge and a lipophilic tail group.

The term “hexagon” or “hexagonal” is to be interpreted broadly to refer to a six-sided polygon, which can be but is not limited to a regular hexagon.

The term “osmolality” is to be interpreted broadly to refer to a measure of the osmotic pressure of dissolved solute particles in an aqueous solution. Said dissolve solute particles includes both ionized and non-ionized molecules.

The term “organic solvent” is to be interpreted broadly to refer to a carbon containing liquid which can solubilize lipophilic and/or hydrophilic compounds.

The term “quaternary ammonium salt” is to be interpreted broadly to include a compound having a positively charged nitrogen atom covalently bonded to four independently selected functional groups including Hydrogen (which can be different or the same), and whose charge is balanced by an anionic counterion.

As used herein, the term “microporous” is used to describe materials or particles having pore size of less than 2 nm and “macroporous” is used to describe materials or particles having pore size of larger than 50 nm. The term “mesoporous” as used herein is the conventionally accepted reference to materials with pore dimensions between that of macroporous particle and microporous particle, i.e. between 2 and 50 nm pore size. The definition of the words “microporous”, “mesoporous” and “macroporous” is consistent with the International Union of Pure and Applied Chemistry notation.

The term “monodispersed” as used herein is to be interpreted broadly to refer to a population of particles which are substantially similar or identical in size, shape and geometry.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Detailed Disclosure of Embodiments

Exemplary, non-limiting embodiments of silica particles will now be disclosed.

The disclosed silica particles may be hexagonal platelets, spheres or torus-like particles. The silica particles may be plate-like or disk-like structures having a generally hexagonal shape. In cases where the silica particles are microporous or mesoporous, the hexagonal shape is intended to characterize the macrostructure of the silica particles and not the shape of the microporous or mesoporous structures that may be integrally formed within the silica particles.

The hexagonal silica platelets may be substantially monodisperse, i.e., these platelets may be substantially uniform in size, width, length and/or thickness. The disclosed silica platelets may have a length dimension of around 50 to 1000 nm, e.g., around 50, 60, 70, 80, 90, 100, 150, 200, 250, 300, 350, 400, 450, 500, 550, 600, 650, 700, 750, 800, 850, 900, 950, or 1000 nm. The disclosed silica platelets may have a thickness of around 1 to 100 nm, e.g. around 1, 5, 10, 15, 20, 30, 40, 50, 60, 70, 80, 90 or 100 nm. The standard deviation in a dimension measurement of a substantially monodisperse population of silica platelets may be less than ±10%, ±8%, ±6%, ±4% or ±2%.

The disclosed silica platelets may each have an aspect ratio defined by the ratio of the surface area of the silica particle to the volume of the silica platelets of around 1:2 to 1:50 nm, e.g. around 1:2, 1:4, 1:6, 1:8, 1:10, 1:15, 1:20, 1:25, 1:30, 1:35, 1:40, 1:45 or 1:50 nm. The aspect ratio may be in a range having an upper and lower limit selected from the ratios disclosed herein. The disclosed silica platelets may each have a measured BET surface area of around 300 m²/g and 1000 m²/g, e.g. around 300, 320, 340, 360, 380, 400, 420, 440, 460, 480, 500, 520, 540, 560, 580, 600, 620, 640, 660, 680, 700, 710, 720, 730, 740, 750, 760, 770, 780, 790, 800, 820, 840, 860, 880, 900, 920, 940, 960, 980 or 1000 m²/g. The surface area may be in a range having an upper and lower limit selected from the values disclosed herein.

The silica particles may have smooth surfaces in which the word “smooth” is to be broadly interpreted according to the definition above. The silica particles may have porous structures. The silica particles may be arranged in a substantially planar, tessellated array.

The disclosed method may comprise forming silica nanoparticles in an aqueous medium, the method comprising the steps of: (a) providing a surfactant solution comprising a cationic surfactant in an aqueous medium; and (b) mixing a silane source with the surfactant solution from step (a) under pH conditions of about pH 5 to 8 for forming said silica nanoparticles.

In embodiments, the mixing step (b) may be performed under pH conditions of about pH 5 to 8, e.g. pH 5.2, 5.4, 5.6, 5.8, 6.0, 6.2, 6.4, 6.5, 6.6, 6.8, 6.9, 7.0, 7.1, 7.2, 7.3, 7.4, 7.5, 7.6, 7.7, 7.8, 7.9 or 8.0. In an embodiment, the mixing step (b) may be performed under pH conditions of about pH 6.5 to 7. In a preferred embodiment, the mixing step (b) may be performed under neutral pH conditions of about pH 7. In another preferred embodiment, the mixing step may be performed under pH conditions of about pH 6.5.

In the disclosed method, the mixing step (b) may comprise physical agitation of the mixture comprising the silane source and the surfactant. In one embodiment, the physical agitation comprises submitting said mixture to a vortex. The physical agitation may comprise subjecting the mixture to stirring means, e.g., a magnetic stirrer.

The mixture of step (b) may exhibit an osmolality of not more than 500 mOsm/L. The osmolality may be from about 0 to 500 mOsm/L, e.g., 0, 5, 10, 20, 30, 40, 50, 100, 150, 200, 250, 300, 350, 400, 450, 500 mOsm/L, or may be in a range having limits selected from any two values disclosed herein, e.g., 0 to 500 mOsm/L, 50 to 500 mOsm/L, 100 to 500 mOsm/L, etc. In embodiments, the reaction mixture is substantially devoid of other reactants or charged species apart from the surfactant and the silane source. Advantageously, this means that the reaction mixture may have a low osmolality of not more than 500 mOsm/L.

The disclosed mixing step (b) may be performed at a temperature of between about −10° C. to about 40° C., e.g., −10, −5, 0, 5 10, 12, 14, 16, 18, 20, 22, 24, 25, 26, 28, 30, 32, 34, 36, 38, or 40° C. In one embodiment, the mixing step (b) may be carried out at around 0° C. to about 30° C. In another embodiment, the mixing step (b) may be carried out at 0° C. In another embodiment, the mixing step (b) may be carried out at about 25° C. Advantageously, spherical silica particles may be formed when the mixing step (b) is performed at about 25° C. The disclosed method may be performed at relatively mild temperature conditions including ambient temperature conditions. This may also advantageously remove the need for any cooling/heating steps to be performed.

The disclosed aqueous medium of step (a) may be substantially free of organic solvent. In embodiments, the aqueous medium comprises not more than 5 wt. %, not more than 4 wt. %, not more than 3 wt. %, not more than 2 wt. % or preferably, not more than 1 wt. % organic solvent. In an embodiment, the aqueous medium may be completely free of organic solvent. This may advantageously reduce the amount of waste that needs to be separated from the product at the end of the reaction. More advantageously, the total absence of an organic solvent in the aqueous medium may eliminate the need for a separation step to remove or recover the organic solvent from the reaction product.

The cationic surfactant may be a quaternary ammonium salt of the general chemical formula N+R1R2R3R4, wherein each of R1, R2, R3 and R4 are independently selected from: hydrogen, aliphatic C1-6 alkyl, aliphatic C6-22 alkyl, wherein at least two or more of R1, R2, R3 and R4 are aliphatic C6-22 alkyl, e.g., C6-C20, C6-C18, C6-16, or C6-10 saturated alkyl. In embodiments, R1, R2, R3 and R4 are independently selected from: hydrogen, aliphatic C1-6 alkyl, aliphatic C6-22 alkyl, wherein at least two or more of R1, R2, R3 and R4 are aliphatic C6-22 alkyl, e.g., C6-C20, C6-C18, C6-16, or C6-10 saturated alkyl and the remaining of R1, R2, R3 and R4 are aliphatic C1-6 alkyl, e.g. C1-5, C1-4, C1-3, C1-2 saturated alkyl.

In embodiments, the counter-ion of the quaternary ammonium salt may be a hydrolysable group selected from acetate, carbonate, oxalate, phosphate, chloride and bromide. In one embodiment, the counter-ion is phosphate.

In one embodiment, the cationic surfactant is didodecyldimethylammonium phosphate. Advantageously, the used of this surfactant may result in the formation of monodispersed silica nanoparticles. More advantageously, the use of this surfactant surprisingly resulted in the formation of hexagonal particles with smooth or microporous surfaces.

In embodiments, the cationic surfactant as used herein is provided in an amount of around 0.1 to 10 wt. %, e.g. 0.1, 0.5, 1, 1.5, 2, 2.5, 3, 3.5, 4, 4.5, 5, 5.5, 6, 6.5, 7, 7.5, 8, 8.5, 9, 9.5 or 10 wt. % of the surfactant solution of step (a). In an embodiment, the amount of cationic surfactant provided in the surfactant solution of step (a) is 1 wt. %. Without being bound by theory, the inventors have surprisingly found that when providing the surfactant in the disclosed amounts, it is possible to obtain substantially monodisperse/uniform and hexagonally shaped silica platelets.

The disclosed surfactant solution may comprise only a single type of surfactant. In an embodiment, the surfactant solution of the disclosed method does not contain more than one type of surfactant. In another embodiment, the surfactant solution disclosed herein may only contain one type of cationic surfactant. In an embodiment, the surfactant solution does not contain an anionic surfactant, a non-ionic surfactant and/or an amphoteric surfactant. In an embodiment, the surfactant solution of the disclosed method may comprise only didodecyldimethylammonium phosphate as the only single surfactant.

The disclosed aqueous medium may be selected from water or a salt solution comprising said counter-ion as described above.

The silane source as disclosed herein may be a substituted or unsubstituted silane. In the case of a substituted silane, the substituents may not constitute bulky or reactive groups. In embodiments, the silane source is selected from tetraalkyl silicate, tetraalkoxysilane, organotrialkoxysilane or diorganodialkoxysilane. In preferred embodiments, the silane source is selected from tetraethylorthosilicate (TEOS) or tetramethylorthosilicate (TMOS). In an embodiment, the silane source is TMOS. More than one silane compound may be used.

The disclosed silane source may be provided in an amount of from about 0.1 vol. % to about 20 vol. %, e.g. 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19 or 20 vol. % of the said surfactant solution. In an embodiment, the disclosed cationic surfactant may be provided in volumetric excess to the disclosed silane source. In an embodiment, the silane source may be provided in an amount of from 0.1 vol. % to about 2 vol. % of the said surfactant solution. In embodiments, the surfactant solution may be provided in the mixture of step (b) in an amount of more than 80 vol. %, more than 85 vol. %, more than 90 vol. % or more than 95 vol. %. In embodiments, the surfactant solution may be provided in the mixture of step (b) in an amount from about 80.0 vol. % to about 99.9 vol. %, e.g. 81.0, 82.0, 83.0, 84.0, 85.0, 86.0, 87.0, 88.0, 89.0, 90.0, 91.0, 92.0, 93.0, 94.0, 95.0, 96.0, 97.0, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8 or 99.9 vol. %. Advantageously, the used of the described amount of surfactant solution may result in the formation of monodispersed silica nanoparticles. More advantageously, the use of described amount of surfactant solution surprisingly resulted in the formation of hexagonal silica particles having smooth surfaces.

The disclosed method may further comprise a step of allowing the mixture obtained from step (b) to stand from about 15 to 20 hours. In embodiments, the mixture obtained from step (b) may be allowed to stand for about 15, 16, 17, 18, 19 or 20 hours. In an embodiment, the mixture obtained from step (b) may be allowed to stand for about 16 hours. When allowing the mixture to stand, the method may optionally comprise continuous or periodic physical agitation of the mixture.

In embodiments, the step of allowing the mixture obtained from step (b) to stand for the defined period is performed at a temperature of between about −10° C. to about 40° C., e.g., −10, −5, 0, 5 10, 12, 14, 16, 18, 20, 22, 24, 26, 28, 30, 32, 34, 36, 38, or 40° C. In one embodiment, this step may be carried out at around 0° C. to about 16° C. The disclosed step may be performed at relatively mild temperature conditions including ambient temperature conditions. This may also advantageously remove the need for any cooling/heating steps to be performed.

The disclosed method may be a one-pot synthesis.

The disclosed method may comprise dissolving the surfactant in an aqueous solvent to provide a surfactant solution having a desired concentration of surfactant (e.g., from about 0.1 to 10 wt. %). The diluted surfactant solution may be added to a reactor containing the silane compound batchwise or continuously. The surfactant solution may be cooled to 0° C.-25° C. prior to addition to the silane. Advantageously, cooling the surfactant solution to the disclosed temperature range may allow for good control of the micellar geometries and reaction kinetics which are vital for controlling the morphology of the silica nanoparticles.

In an embodiment, the disclosed method may comprise (a) providing a surfactant solution consisting of 1 wt. % didodecyldimethylammonium phosphate in the aqueous medium; and (b) mixing TMOS with the surfactant solution from step (a) under pH condition of about pH 7 at a temperature of about 0° C. for forming hexagonal silica platelets.

In another embodiment, the disclosed method may comprise (a) providing a surfactant solution consisting of 1 wt. % didodecyldimethylammonium phosphate in the aqueous medium; and (b) mixing TMOS with the surfactant solution from step (a) under pH condition of about pH 7 at a temperature of about 25° C. for forming spherical silica nanoparticles.

In an embodiment, the disclosed method may comprise (a) providing a surfactant solution consisting of about 1.5 to 2 wt. % didodecyldimethylammonium phosphate in the aqueous medium; and (b) mixing TMOS with the surfactant solution from step (a) under pH condition of about pH 6.5 at a temperature of about 0° C. for forming torus-like silica nanoparticles.

In one embodiment, a 1 wt. % surfactant solution is added to the silane in volumetric excess (e.g., 99-90:1-10 vol. %) under constant physical agitation. The agitation may be applied for a period of time necessary to achieve a homogeneous mixture. The mixture may be left to stand for a period of time necessary for the formation of the hexagonal silica particles.

The disclosed silica suspension may comprise a plurality of hexagonally shaped silica platelets. In an embodiment, the silica suspension may comprise hexagonally shaped silica platelets that are substantially monodisperse. In embodiments, the silica suspension may comprise hexagonally shaped silica platelets having a width dimension from around 50 to 2000 nm, e.g. 50, 60, 70, 80, 90, 100, 120, 140, 160, 180, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900 or around 2000 nm. In another embodiment, the hexagonally shaped silica platelets may be suspended in an aqueous medium. In an embodiment, the aqueous medium may comprise at least one cationic surfactant. The presence of the hexagonal shaped silica nanoparticles in the disclosed silica suspension may advantageously modify the surface properties of the suspension. More advantageously, the hexagonal shaped silica nanoparticles surprisingly modify the surface properties of the suspension without affecting the viscosity of the suspension significantly.

In an embodiment, the aqueous medium in the disclosed silica suspension of claim may not contain an organic solvent as described above.

In an embodiment, the silica platelets in the silica suspension may be disposed on the cationic surfactant. In embodiments, the cationic surfactant in the silica suspension may be as described above.

In an embodiment, the disclosed silica platelet may be prepared by reacting a silane source with a volumetric excess of a surfactant under pH of about 7 in the presence of an aqueous solution.

In an embodiment, the disclosed silica platelet may be obtainable by a method as disclosed herein.

In an embodiment, the disclosed cosmetic composition may comprise a silica suspension or a silica platelet as disclosed herein.

In an embodiment, the disclosed method of providing UV shielding properties to a composition may comprise adding a silica suspension or a silica platelet as disclosed herein to said composition. In an embodiment, the composition is a cosmetic composition formulated for topical administration.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a SEM image showing the hexagonal silica particles prepared according to the method disclosed herein. The scale bar represents 1 μm.

FIG. 2 is a 3-dimensional AFM image showing the hexagonal silica particles prepared according to the method disclosed herein.

FIG. 3 shows (a) a planar AFM image, (b) the corresponding topographical profile of the hexagonal silica particles along a linear direction shown in (a), (c) a magnified planar AFM image of (a), and (d) the corresponding topographical profile of the hexagonal silica particles along a linear direction shown in (c).

FIG. 4 is a graph showing the viscosity profiles against the shear rate of 0.12, 0.5 and 1 wt. % of hexagonal silica plates in water according to the present invention.

FIG. 5 shows (a) the effect of mixing blood with thrombin and (b) the interfacial stabilizing effect when mixing blood with a 0.1 wt. % thrombin functionalized hexagonal silica nanoparticles in water.

FIG. 6 shows the UV/VIS absorbance spectrum of 1 wt. % silica plates and 1 wt. % TiO₂ (cosmetic UV grade) films deposited on quartz substrates,

### EXAMPLES

Non-limiting examples of the invention and a comparative example will be further described in greater detail by reference to specific Examples, which should not be construed as in any way limiting the scope of the invention.

### Example 1—Preparation of Silica Nanoparticles

1 wt. % of didodecyldimethylammonium phosphate solution is prepared at pH 7. The solution is chilled in an ice bath for 15 min prior to mixing with tetramethylorthosilicate (TMOS). After mixing TMOS with the didodecyldimethylammonium phosphate solution, the mixture is vortexed (10,000 rpm, 30 seconds) and incubated overnight for 16 hours at 0° C.

After incubation, the mixture containing the silica nanoparticles formed is centrifuged (15,000 g, 5 min) and the pellet is recovered and washed with deionized water three times to remove residual surfactant and unreacted TMOS. The remaining silica nanoparticles are dried and characterized.

### Example 2—Characterizations of Silica Nanoparticles

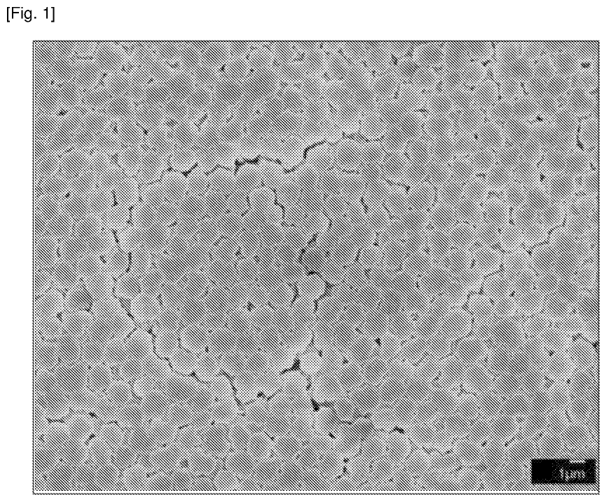

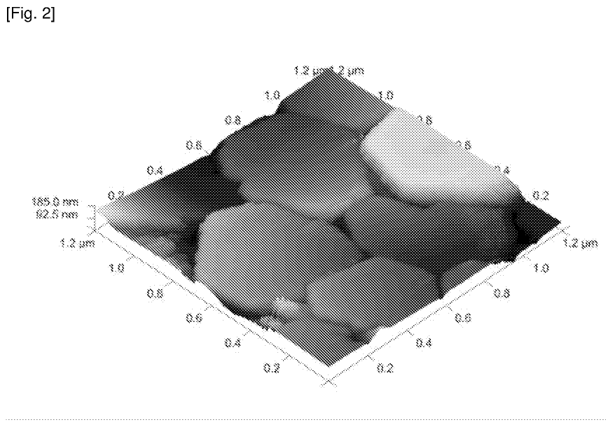

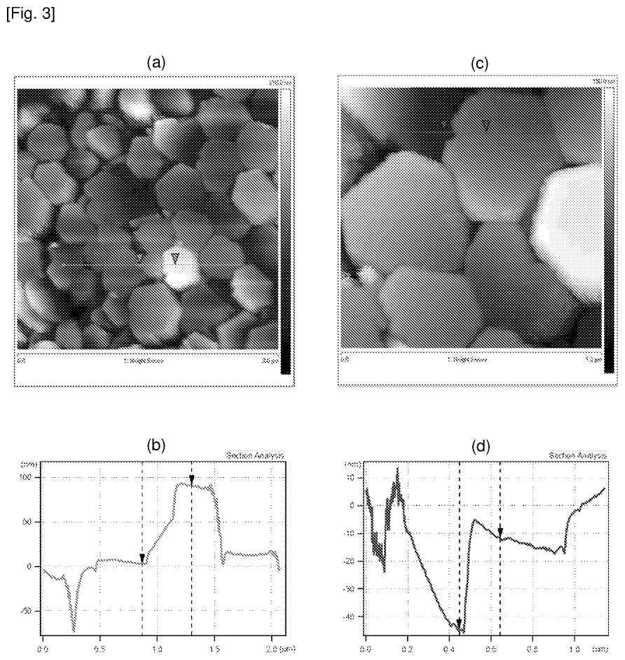

The dried silica nanoparticles are characterized with scanning electron microscopy (SEM) and atomic force microscopy (AFM). FIG. 1 shows the SEM image of the silica nanoparticles with uniform particle size and morphology. FIG. 2 shows the three-dimensional AFM image of the silica nanoparticles. It can be seen from FIGS. 1 and 2 that the silica nanoparticles are hexagonal in shape. In addition, the topographical image of FIG. 3 shows that the silica nanoparticle has a measured thickness of about 40 nm.

In addition, the BET surface area of the silica nanoparticles is measured to be 768 m²/g.

Furthermore, the surface area-to-volume aspect ratio of the silica nanoparticles is calculated based on the length, width and thickness of the nanoparticles measured using the AFM. The surface area-to-volume aspect ratio of the silica nanoparticles is calculated to be about 1:20 nm.

### Example 3—Viscosity Profiles of Hexagonal Silica Nanoparticles Suspensions

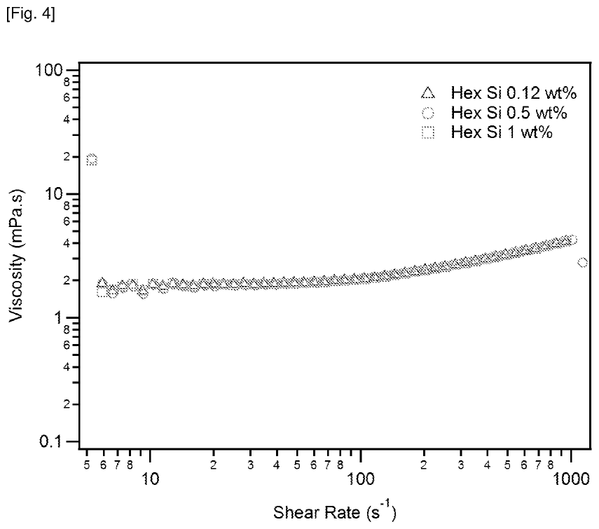

The dried silica nanoparticles prepared according to Example 1 are re-suspended in water to obtain samples with 0.12, 0.5 and 1 wt. % hexagonal silica nanoparticles in water. The viscosity profiles of these samples are measured against the shear rate at 25° C. (DHR-3, TA Instruments) and the results are shown in FIG. 4.

Surprisingly, the disclosed hexagonal silica platelets do not affect the viscosity of the aqueous solutions.

### Example 4—Interfacial Stabilizing Effect of Hexagonal Silica Nanoparticles Functionalized with Thrombin

10U of bovine thrombin is incubated with 1 ml of 0.1 wt. % silica hexagonal silica nanoparticles, prepared according to Example 1, in the presence of 0.001 wt. % NHS-silane under mixing for 2 hours at 4° C. The resulting mixture is spin-washed with cold sterile DI water twice. The resultant thrombin functionalized hexagonal silica nanoparticles are re-suspended in 1 ml of sterile cold DI water.

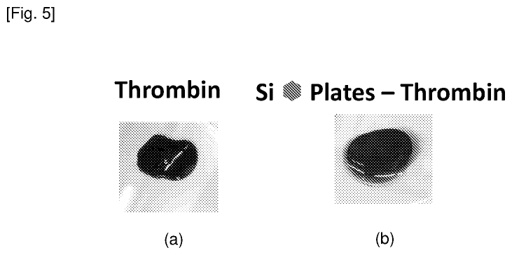

FIG. 5(a) shows that when blood is mixed with thrombin, a homogenous phase is resulted. In comparison, when blood is mixed with 0.1 wt. % aqueous thrombin functionalized hexagonal silica nanoparticles solution in ratio of 1:1, two discrete phases are formed as shown in FIG. 5(b), surprisingly, demonstrating the interfacial stabilizing effect between blood and an aqueous phase comprising thrombin functionalized hexagonal silica nanoparticles.

### Example 5—UV/VIS Absorbance Spectrum of Silica Nanoparticles

Silica plates of 1 μm in diameter, 40 nm in thickness are synthesized by drop-wise addition of TMOS to 1 wt. % of didodecyldimethylammonium phosphate solution (pH 7) at 0° C. under vigourous mixing until the final concentration of TMOS reaches 0.3 wt. %. The osmolarity of the mixture is about 100 mOsm/L. The mixture is incubated overnight at 0° C. After incubation, the silica plates formed are recovered by washing with deionized water. The recovered silica plates are further cleaned by calcination at 500° C. The cleaned silica plate is then re-suspended in water to obtain a concentration of 1 wt. %.

A silica film is prepared by drop casting the 1 wt. % silica plate suspension on a quartz substrate. The dried film is subsequently subjected to UV/VIS absorbance measurement. To assess the UV/VIS absorbance characteristic of the silica plate, the UV/VIS absorbance of a TiO₂ film, prepared by dry casting 1 wt. % TiO₂ suspension on another quartz substrate, is measured.

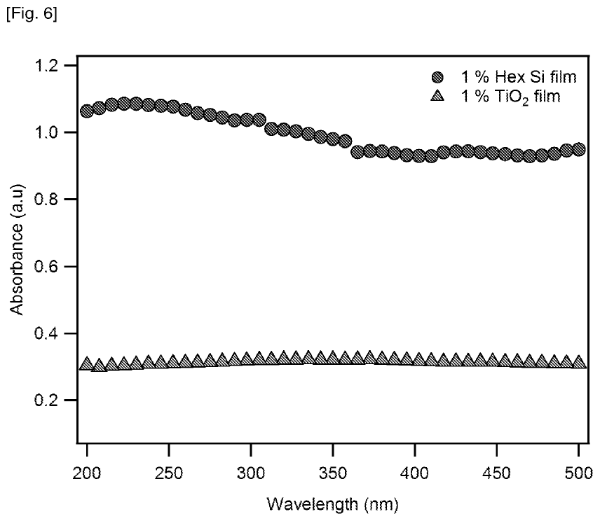

FIG. 6 shows a comparison of the UV/VIS absorbance spectrum of 1 wt. % silica plates and 1 wt. % TiO₂ (cosmetic UV grade) films deposited on quartz substrates. As demonstrated in FIG. 6, unlike traditional fumed silica which is widely understood to be transparent to UV, the silica plates of the present disclosure unexpectedly exhibit about three times the absorbance of cosmetic grade TiO₂ UV filters. Advantageously, the silica plates disclosed herein may potentially be used in cosmetic formulations as whitening agents or as pigments for UV blocking or absorption compositions.

### INDUSTRIAL APPLICABILITY

The disclosed methods for preparing silica particles are advantageously simple (one-pot synthesis) and is capable of providing silica particles that are substantially uniform in size and distribution. The disclosed methods can also be carried out at mild temperature and pH conditions, allowing such methods to be readily scaled up for industrial processes.

The hexagonal silica plates may advantageously be used as deposition aid. Surprisingly, the silica plates may be uniformly deposited on a surface, providing more than 99.5% coverage of the surface.

The hexagonal silica platelets prepared by the disclosed method may advantageously be used as a modifier of the surface properties of a solution or suspension. Surprisingly, the disclosed hexagonal silica platelets may change the surface properties of a liquid without significantly affecting the viscosity of the liquid.

The disclosed hexagonal silica platelets may be post-functionalized for used in drug delivery. Furthermore, functionalized hexagonal silica platelets may be used as support for catalysts or enzymes.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of forming silica nanoparticles in an aqueous medium, the method comprising:
(a) providing a surfactant solution comprising a cationic surfactant in the aqueous medium; and
(b) mixing a silane source with the surfactant solution from operation (a) under pH conditions of about pH 5 to 8 for forming said silica nanoparticles,
wherein said cationic surfactant is a quaternary ammonium salt comprising at least two independently selected C₆-C₂₂ alkyl groups and covalently bonded to the positively charged nitrogen atom; and wherein said quaternary ammonium salt has a counter-ion selected from acetate, carbonate, oxalate, phosphate, chloride or bromide, and
wherein operation (b) is performed at a temperature of between about 0° C. to about 25° C.

2. The method of claim 1, wherein the surfactant solution consists essentially of a single type of cationic surfactant.

3. The method of claim 1, wherein the mixture of operation (b) has an osmolality of not more than 500 mOsm/L.

4. The method of claim 1, wherein said aqueous medium does not contain an organic solvent.

5. The method of claim 1, wherein said cationic surfactant is didodecyldimethylammonium phosphate.

6. The method of claim 1, wherein said cationic surfactant is provided in an amount of around 0.1 to 10 wt. % of said surfactant solution.

7. The method of claim 1, wherein said aqueous medium is selected from water or a salt solution comprising said counterion.

8. The method of claim 1, wherein said slime source is selected from tetraalkyl silicate, tetraalkoxysilane, organotrialkoxysilane or diorganodialkoxysilane.

9. The method of claim 1, wherein said silane source is selected from tetraethylorthosilicate (TEOS) or tetramethylorthosilicate (TMOS).

10. The method of claim 1, wherein said silane source is provided in an amount of from about 0.1 vol. % to about 20 vol. % of the said surfactant solution.

11. The method of claim 1, wherein said surfactant is provided in volumetric excess to said silane source or wherein said silane source is provided in an amount of 0.1 to 2 vol. % based on said surfactant solution.

12. The method of claim 1, the method further comprising an operation of allowing the mixture obtained from operation (b) to stand from 15 to 20 hours at a temperature of between about 0° C. to about 30° C.

13. (canceled)

14. A silica suspension comprising a plurality of hexagonally shaped silica platelets, said platelets being substantially monodisperse and having a width dimension from around 50 to 2000 nm, said platelets being suspended in an aqueous medium, wherein said aqueous medium comprises a single type of cationic surfactant, wherein the silica platelets do not have a mesoporous structure.

15. The silica suspension of claim 14, wherein said aqueous medium does not contain an organic solvent.

16. The silica suspension of claim 14, wherein said silica platelets are disposed on said cationic surfactant.

17. The silica suspension of claim 14, wherein said cationic surfactant is a quaternary ammonium salt comprising at least two C₆-C₂₂ alkyl groups independently and covalently bonded to the positively charged nitrogen atom; and wherein said quaternary ammonium salt has a counterion selected from acetate, carbonate, oxalate, phosphate, chloride and bromide.

18. The silica suspension of claim 17, wherein said cationic surfactant is didodecyldimethylammonium phosphate.

19. The silica suspension of claim 14, wherein the silica platelets have surface area-to-volume ratio of between 1:2 nm and 1:50 nm or 1:20 nm.

20. The silica suspension of claim 14, wherein the silica platelets have BET surface area of between 300 m²/g and 1000 m²/g.

21. A hexagonally shaped silica nanoparticle having a width dimension from around 50 to 2000 nm, wherein the nanoparticle does not have a mesoporous structure.

22. A silica nanoparticle obtainable by a method of forming silica nanoparticles in an aqueous medium, the method comprising:
(a) providing a surfactant solution comprising a cationic surfactant in the aqueous medium; and
(b) mixing a silane source with the surfactant solution from operation (a) under pH conditions of about pH 5 to 8 for forming said silica nanoparticles,
wherein said cationic surfactant is a quaternary ammonium salt comprising at least two independently selected C₆-C₂₂ alkyl groups and covalently bonded to the positively charged nitrogen atom; and wherein said quaternary ammonium salt has a counter-ion selected from acetate, carbonate, oxalate, phosphate, chloride or bromide, and
wherein operation (b) is performed at a temperature of between about 0° C. to about 25° C., wherein the nanoparticle is hexagonally shaped and does not have a mesoporous structure.

23. A cosmetic composition comprising a silica suspension comprising a plurality of hexagonally shaped silica platelets, said platelets being substantially monodisperse and having a width dimension from around 50 to 2000 nm, said platelets being suspended in an aqueous medium, wherein said aqueous medium comprises a single type of cationic surfactant, wherein the silica platelets do not have a mesoporous structure, or hexagonally shaped silica nanoparticles having respective width dimensions from around 50 to 2000 nm, wherein each nanoparticle does not have a mesoporous structure.

24. A method of providing UV shielding properties to a composition, comprising adding a silica suspension comprising a plurality of hexagonally shaped silica platelets, said platelets being substantially monodisperse and having a width dimension from around 50 to 2000 nm, said platelets being suspended in an aqueous medium, wherein said aqueous medium comprises a single type of cationic surfactant, wherein the silica platelets do not have a mesoporous structure, or hexagonally shaped silica nanoparticles having respective width dimensions from around 50 to 2000 nm, wherein each nanoparticle does not have a mesoporous structure.

25. The method of claim 23, wherein said composition is a cosmetic composition formulated for topical administration.
